SEQUENCE LISTING (4 sequences)

SEQ ID NO: 1 - DNA, 5615 bp, synthetic construct
  source 1..5615: mol_type = other DNA
accggtatcacgtgccatcgatcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattgcggccgcataacttcgtataatgtatgctatacgaagttatccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcggccataacttcgtataatgtatgctatacgaagttatgcggccgctttattaattaagccctgcaggttgtttaaacgaaccgcgggccctttagactcgagcggccgccactgtgctggatatcaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgttgatatctgcagaattccaccacactggactagtggatccggtaccaagcttacctagccagcttgggtctccctataggcgcgccaatatcgataagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaatt

SEQ ID NO: 2 - DNA, 6416 bp, synthetic construct
  source 1..6416: mol_type = other DNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggctagcttaggggctgtacagggtgcgctcgggtttctgaactctcagatagtggggttcccgggaaagggcctatatttgcggtccaacttaggcgtaaacctcggtactacctactcagacccaccccgcgcggggtaagtatagcactgctcccagctggttcttggcgttctacgcagccaggtgttgcttaacagttgtctggtagcacaaaagtattaccgtagtcctagaagcccggcagagttagttcgaacctagtaccacaagtaagacaggacgccagtagctaccggagcttaggtcgcagtcagttcggtaacggagagaccctgcggcgtacttaattgtacacgtaaaacgcgcccaagtgacgccaggcaagtctcagcaggttcccgtgttagctcgagggtaaagctacaagccgattgaacgtaggttgggggcttcaaatcgtcgaggaccccacagtacctcggagaccaagtagggcaccctatagtaggcctcagaactatttcgaggggcgagccctgctcgtctcttctgcgggtaacttaacacgctagggacgtggagtcgattcgctcggtagttagaaatcaaagattcggagttctgtctggagggtgaatctaacggtgcgtatctcgattgctcagtcgcttttcgtactgcgcgaaagttcgtaccgctgcttcacttggttccgaagcctgtcctgtgatataaatccaaactagagcggggctcttgagctttggagttgtaaatatctaatactccaatcggcttttacgtgcaccaccgcgggcggctgacgagggtctcacaccgagaaacaagacagttccgggctggaagtagcggtggctaaggaagacgcctggtacagcaggactgtaaaacgggtacaaaggcaagctcctcacttgggtgaaccgaaacgcggtatcaaggttactttttggatacctgaaacaaatcccgtagtagtccttaggttaacgagtctatcacccctagggccgctatctggaaatagacgccaagttcaatccgtattccgacgtacggtagaacagtgtgggtgagacgtgcttgctttataccctacgcaggctggaccgaggtccgcaaggcgcggcggtgcacaagcaattgacaactaaccaccgtgtattgcttgtagtaccagggactttaagccgagtcagtagtcgtcgcaatacagagtttaccggctcttgccgtaactgacaaactgtgatccaccacaagtcaagcgacttgcctcttagacacgccgttagagtaattgtataaactttgcgcggcttgactacgactcgttcagtcacgtccgagggcacaatcctattccgctttgtgtattcagctatcttctaccgctccccggaagttaagtaggtcgtgaggcacgaggaggctctcgttgctcccgtgggcgttcaagcttccccttgataaagcaccccgctctcgcgaagcagagaggacgccttctgaattgtgctatccctcgaccttatcaaagcttgctaccaataattaggattattgccttgcgacagacctcctactcagactgcctcagcttgagctagtcagtgagcgataagcttgacccgctttctagggacgcgagtacgtgaactagggctccggacagggctatatactcgagtttgatctcgccccgacaactgcaaacctcaacttttttagatagtatagttagccgaagttgcacgaggtgccgtccgcggactgctccccgggtgtggctccttgctctgacaacgtgcaacccctatcgcgctcgcttgtttctgcggacggtgttgtcctgctagtttgggcgtatttcccttgtaggtgtgaaaccacttagcttcgcgccgtagtcctaaaggaaaacctgtagactttgtttcgggtagcaccaggaatctgaaccgtgtaagtgtagacgtggcgcgcacgtgccttaatctccggttcgtactaggggtgtagctgcgtactacgttgacacacctacactgctcgaagtaaatatacgaagcgcgcggcctggccggagccgttccggctcgtgacgtgttcgtttactgttaattggtggcagctaagcaatatcgtagtccgtcaaattcagccctgttatccccggcgttgtgtatcaagtagcgtagaactggattgactgtttgacgaatcctgctgatcggtacggtgaccgagaatctgtcgggctgtatcactaatactttccaaacgccccgtatcggtactgaacgaatcggtacacgctcccgtctttgaaaacggctaatcgtacaagtggacaggtagtaggtacgggcctctaatagctccaacactctacgccctcttcaagagctagaagggcaccctccagttggaaagggaattatttcgtaaggcgagccgctaccgtgcttcgtacggaagagtgtttaaaccaacacgattggaagtaggaatagtttcgaaccacggttactaatcctaataacggaacgctgtctgaagggtaagtgtcagcgagtgtaactcggtaagctacccagtagtcgaactgggcgagacaacccggcgctagtacactcaatcccgtggcctgacgcgagctatcagcttagactagggcgggggtgttgacgtttggggttgaataaatctattgtactaatcggcttcaacgtgccccacgggtggcacctcaggaggggcccacagcgaggaagtaaactgttattcgtcggcggtagtggtagctaattgtattccttgccactacaatagtatctaagccgtgtagtaggaagctccacactttagtgaatcggtacgcggcttcagaataccgttttggctacctgttactaagccgctcgtggttttcagatagcgtgcacgtagggttgcaccgcacgcgtgtagaattacgtggcaggcgccagtacgattccacgaccgacgtacgattcaactgtacggacgtgacgagcttcttttgtatacttcgcccgccggaccggcctcgtggtagggtagctgcggctaagcttgtaacaattaacgagtgtgtactcgttttctgctctcacagttaaagtcgggagaataggagccgctacacacaatttaggtagccttgacctaactgagatactgcgctagacgactaccgctccctctgggccttagatagccggatacagtgactttgaaaggtttgtggggtacagctgtaacttgcttagctgcgtgtgggggaaggaacttttgcgtgttagtgtattgacccgtgtattacgcgtacgggtagattgtataggagagctgtccaggtcaagttctcgaccttctcgtgggaggtgaaccagttcactataggacgcttccgttcgagcgtagcactaagtacgccctcccgcttctggtaatcttcagatatcgagctagcactctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 9052 bp, synthetic construct
  source 1..9052: mol_type = other DNA
ccaatgatatcgaattcgcggccgcactagtgtttaaacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaatcaaccactttgtacaagaaagctgggtttatgtttcaggttcagggggaggtgtgggaggttttttaaagcaagtaaaacctctacaaatgtggtatggctgattatgatcatgaacagactgtgaggactgaggggcctgaaatgagccttgggactgtgaatcaatgcctgtttcatgccctgagtcttccatgttcttctccccaccatcttcatttttatcagcattttcctggctgtcttcatcatcatcatcactgtttcttagccaatctaaaactccaattcccatagccacattaaacttcattttttgatacactgacaaactaaactctttgtccaatctctctttccactccacaattctgctctgaatactttgagcaaactcagccacaggtctgtaccaaattaacataagaagcaaagcaatgccactttgaattattctcttttctaacaaaaactcactgcgttccaggcaatgctttaaataatctttgggcctaaaatctatttgttttacaaatctggcctgcagtgttttaggcacactgtactcattcatggtgactattccagggggaaatatttgagttcttttatttaggtgtttcttttctaagtttaccttaacactgccatccaaataatcccttaaattgtccaggttattaattccctgacctgaaggcaaatctctggactcccctccagtgccctttacatcctcaaaaactactaaaaactggtcaatagctactcctagctcaaagttcagcctgtccaagggcaaattaacatttaaagctttccccccacataattcaagcaaagcagctgctaatgtagttttaccactatcaattggtcctttaaacagccagtatctttttttaggaatgttgtacaccatgcattttaaaaagtcatacaccactgaatccattttgggcaacaaacagtgtagccaagcaactccagccatccattcttctatgtcagcagagcctgtagaaccaaacattatatccatcctatccaaaagatcattaaatctgtttgttaacatttgttctctagttaattgtaggctatcaacccgctttttagctaaaacagtatcaacagcctgttggcatatggttttttggtttttgctgtcagcaaatatagcagcatttgcataatgcttttcatggtacttatagtggctgggctgttcttttttaatacattttaaacacatttcaaaactgtactgaaattccaagtacatcccaagcaataacaacacatcatcacattttgtttccattgcatactctgttacaagcttccaggacacttgtttagtttcctctgcttcttctggattaaaatcatgctcctttaacccacctggcaaactttcctcaataacagaaaatggatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctcagttgcatcccagaagcctccaaagtcaggttgatgagcatattttactccatcttccattttcttgtacagagtattcattttcttcattttttcttcatctcctcctttatcaggatgaaactccttgcatttttttaaatatgcctttctcatcagaggaatattcccccaggcactcctttcaagacctagaaggtccattagctgcaaagattcctctctgtttaaaactttatccatggtagcctgcttttttgtacaaacttgttgatcgcgttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccgtttaaaccctgcaggccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggctagcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggcgcgccaatatcgataagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggccatggtgctgcgccggctgtcacgccaggcctccgttaaggttcgtaggtcatggactgaaagtaaaaaaacagctcaacgcctttttgtgtttgttttagagcttttgctgcaattttgtgaaggggaagatactgttgacgggaaacgcaaaaaaccagaaaggttaactgaaaaaccagaaagttaactggtaagtttagtctttttgtcttttatttcaggtccatgggtgctgctttaacactgttgggggacctaattgctactgtgtctgaagctgctgctgctactggattttcagtagctgaaattgctgctggagaggccgctgctgcaattgaagtgcaacttgcatctgttgctactgttgaaggcctaacaacctctgaggcaattgctgctataggcctcactccacaggcctatgctgtgatatctggggctcctgctgctatagctggatttgcagctttactgcaaactgtgactggtgtgagcgctgttgctcaagtggggtatagattttttagtgactgggatcacaaagtttctactgttggtttatatcaacaaccaggaatggctgtagatttgtataggccagatgattactatgatattttatttcctggagtacaaacctttgttcacagtgttcagtatcttgaccccagacattggggtccaacactttttaatgccatttctcaagctttttggcgtgtaatacaaaatgacattcctaggctcacctcacaggagcttgaaagaagaacccaaagatatttaagggacagtttggcaaggtttttagaggaaactacttggacagtaattaatgctcctgttaattggtataactctttacaagattactactctactttgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagtggatgttgcctttacttctaggcctgtacggaagtgttacttctgctctaaaagcttatgaagatggccccaacaaaaagaaaaggaagttgtccaggggcagctcccaaaaaaccaaaggaaccagtgcaagtgccaaagctcgtcataaaaggaggaatagaagttctaggagttaaaactggagtagacagcttcactgaggtggagtgctttttaaatcctcaaatgggcaatcctgatgaacatcaaaaaggcttaagtaaaagcttagcagctgaaaaacagtttacagatgactctccagacaaagaacaactgccttgctacagtgtggctagaattcctttgcctaatttaaatgaggacttaacctgtggaaatattttgatgtgggaagctgttactgttaaaactgaggttattggggtaactgctatgttaaacttgcattcagggacacaaaaaactcatgaaaatggtgctggaaaacccattcaagggtcaaattttcatttttttgctgttggtggggaacctttggagctgcagggtgtgttagcaaactacaggaccaaatatcctgctcaaactgtaaccccaaaaaatgctacagttgacagtcagcagatgaacactgaccacaaggctgttttggataaggataatgcttatccagtggagtgctgggttcctgatccaagtaaaaatgaaaacactagatattttggaacctacacaggtggggaaaatgtgcctcctgttttgcacattactaacacagcaaccacagtgcttcttgatgagcagggtgttgggcccttgtgcaaagctgacagcttgtatgtttctgctgttgacatttgtgggctgtttaccaacacttctggaacacagcagtggaagggacttcccagatattttaaaattacccttagaaagcggtctgtgaaaaacccctacccaatttcctttttgttaagtgacctaattaacaggaggacacagagggtggatgggcagcctatgattggaatgtcctctcaagtagaggaggttagggtttatgaggacacagaggagcttcctggggatccagacatgataagatacattgatgagtttggacaaaccacaactagaatgcagtgacgtacgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctagagcggccgcaagcttgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 7810 bp, synthetic construct
  source 1..7810: mol_type = other DNA
cactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcggccataacttcgtataatgtatgctatacgaagttatgcggccgctttattaattaagccctgcaggttgtttaaacgaaccgcgggccctttagactcgagcggccgccactgtgctggatatcaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgttgatatctgcagaattccaccacactggactagtatcctgcaggaaggtaccaagacgtcaaggcgcgccaatatcgataagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggccatggtgctgcgccggctgtcacgccaggcctccgttaaggttcgtaggtcatggactgaaagtaaaaaaacagctcaacgcctttttgtgtttgttttagagcttttgctgcaattttgtgaaggggaagatactgttgacgggaaacgcaaaaaaccagaaaggttaactgaaaaaccagaaagttaactggtaagtttagtctttttgtcttttatttcaggtccatgggtgctgctttaacactgttgggggacctaattgctactgtgtctgaagctgctgctgctactggattttcagtagctgaaattgctgctggagaggccgctgctgcaattgaagtgcaacttgcatctgttgctactgttgaaggcctaacaacctctgaggcaattgctgctataggcctcactccacaggcctatgctgtgatatctggggctcctgctgctatagctggatttgcagctttactgcaaactgtgactggtgtgagcgctgttgctcaagtggggtatagattttttagtgactgggatcacaaagtttctactgttggtttatatcaacaaccaggaatggctgtagatttgtataggccagatgattattatgatattttatttcctggagtacaaacctttgttcacagtgttcagtatcttgaccccagacattggggtccaacactttttaatgccatttctcaagctttttggcgtgtaatacaaaatgacattcctaggctcacctcacaggagcttgaaagaagaacccaaagatatttaagggacagtttggcaaggtttttagaggaaactacttggacagtaattaatgctcctgttaattggtataactctttacaagattactactctactttgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagtggatgttgcctttacttctaggcctgtacggaagtgttacttctgctctaaaagcttatgaagatggccccaacaaaaagaaaaggaagttgtccaggggcagctcccaaaaaaccaaaggaaccagtgcaagtgccaaagctcgtcataaaaggaggaatagaagttctaggagttaaaactggagtagacagcttcactgaggtggagtgctttttaaatcctcaaatgggcaatcctgatgaacatcaaaaaggcttaagtaaaagcttagcagctgaaaaacagtttacagatgactctccagacaaagaacaactgccttgctacagtgtggctagaattcctttgcctaatttaaatgaggacttaacctgtggaaatattttgatgtgggaagctgttactgttaaaactgaggttattggggtaactgctatgttaaacttgcattcagggacacaaaaaactcatgaaaatggtgctggaaaacccattcaagggtcaaattttcatttttttgctgttggtggggaacctttggagctgcagggtgtgttagcaaactacaggaccaaatatcctgctcaaactgtaaccccaaaaaatgctacagttgacagtcagcagatgaacactgaccacaaggctgttttggataaggataatgcttatccagtggagtgctgggttcctgatccaagtaaaaatgaaaacactagatattttggaacctacacaggtggggaaaatgtgcctcctgttttgcacattactaacacagcaaccacagtgcttcttgatgagcagggtgttgggcccttgtgcaaagctgacagcttgtatgtttctgctgttgacatttgtgggctgtttaccaacacttctggaacacagcagtggaagggacttcccagatattttaaaattacccttagaaagcggtctgtgaaaaacccctacccaatttcctttttgttaagtgacctaattaacaggaggacacagagggtggatgggcagcctatgattggaatgtcctctcaagtagaggaggttagggtttatgaggacacagaggagcttcctggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattgcggccgcataacttcgtataatgtatgctatacgaagttatccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagct

(19) United States
(12) Patent Application Publication
(10) Pub. No.: US 2026/0234658 A1
(43) Pub. Date: Aug. 13, 2026

(54) COMPOSITION AND METHOD FOR THE PRODUCTION OF POLYOMAVIRAL VECTOR PARTICLES

(71) Applicant: AMARNA HOLDING B.V., Leiden (NL)

(72) Inventors: Ferdy Rudolf VAN DIEMEN, Leiden (NL); Laurens Christiaan DE GIER, Leiden (NL); Petrus Theodorus DE HAAN, Leiden (NL)

(21) Appl. No.: 19/119,154

(22) PCT Filed: Oct. 10, 2023
(86) PCT No.: PCT/NL2023/050525

§ 371 Date: Apr. 8, 2025

(30) Foreign Application Priority Data
Oct. 10, 2022 (NL) 2033264

(51) Int. Cl. C12N 15/86; A61K 39/12; A61K 48/00

(52) U.S. Cl. CPC C12N 15/86; A61K 39/12; A61K 48/0008; C12N 2710/22043; C12N 2710/22052

(57) ABSTRACT

The present invention relates to a composition for use in the production of polyomaviral vector particles, the composition comprising a first DNA construct comprising recombinant DNA and a polyomaviral intergenic region, wherein said first DNA construct does not comprise polyomaviral functional coding sequences and bacterial plasmid sequences, and a second DNA construct comprising polyomaviral functional coding sequences encoding functional polyomaviral capsid proteins, wherein said second DNA construct cannot be encapsidated into the polyomaviral vector particles. The present invention further relates to a method for the production of polyomaviral vector particles using the first and second DNA constructs and the polyomaviral vector particles for use as a medicament, preferably for use in the treatment of genetic disorders and immunity-associated diseases.

### TECHNICAL FIELD

The present invention relates to a composition for use in the production of polyomaviral vector particles and to a method for the production of polyomaviral vector particles. The present invention further relates to polyomaviral vector particles for use as a medicament, preferably for use in the treatment of genetic disorders and immunity-associated diseases.

### BACKGROUND

Over the last decades, much effort has been dedicated to the development of efficient gene or nucleic acid delivery technologies for introduction and proper expression of genes or nucleic acids in target cells. Therapeutic genes or nucleic acids can be used to restore malfunctioning genes to treat genetic disorders, to induce an immune response to treat cancer and infectious diseases or to suppress an immune response e.g. for inducing/restoring immune tolerance to prevent transplant rejection or to treat autoimmune diseases and allergies. The therapeutic genes or nucleic acids can be administered as naked molecules or as nucleic acids packaged in lipid and/or proteinaceous compounds.

Since viruses evolved to deliver and express their genetic information into host target cells, viral vectors are by far the most effective gene delivery vehicles to express self or foreign proteins in vivo. Among the viral vectors currently used for treating genetic disorders, cancer, autoimmune diseases and allergies, and for preventing transplant rejection, replication-defective lentiviral (LV) vectors derived from the human immunodeficiency virus type 1 and replication-defective adeno-associated viral (AAV) vectors derived from adeno-associated virus are the most popular. For both replication-defective vectors it has been shown that they are non-immunogenic or tolerogenic in hosts that are naïve to the cognate virus. LV vectors permanently modify transduced target cells by integrating their viral genomes randomly in the host genome. Since the particles are highly instable LV vectors are mainly used for ex vivo gene replacement therapy to treat blood-related genetic disorders and cancer. The genomes of AAV vectors remain as stable episomes in the nuclei of transduced target cells and since the particles are highly stable AAV vectors are mainly used for in vivo gene therapies. AAV is a primarily human virus that co-replicates with adenoviruses: the causal agents of the common cold. The vast majority of the human population has been exposed to AAV and developed a strong immune memory for the viral capsid proteins. Numerous clinical studies using recombinant AAV vectors indeed confirmed that administration of vector particles elicits innate and adaptive immune responses against the viral and transgene-encoded proteins in the vast majority of treated patients. The immune responses lead to elimination of the transduced cells from the body and decreasing expression levels of the therapeutic transgenes over time, compromising re-administration of the vector. The few treated patients that showed long term transgene expression most likely have never been infected with AAV and thus were immunologically naïve to the AAV vector used in the study. In these patients the treatment of a single dose of an AAV vector may lead to over-expression of the transgene resulting in the development of in some cases severe adverse effects. AAV's immunogenicity and toxicity in humans, and as a result its clinical inefficacy, will remain the major challenges for the approval of new AAV vector-based interventions.

Replication-defective polyomaviral vectors are an attractive alternative to AAV vectors for clinical gene therapy. Polyomavirus strictly replicate in their natural host, where they cause chronic asymptomatic infections. Replication-defective polyomaviral vectors are non-immunogenic in hosts that are immunologically naïve for the cognate polyomavirus. Simian virus 40 (SV40) is a polyomavirus that naturally and strictly infects macaques, where it causes chronic asymptomatic infections. SV40 particles enter infected cells via the caveolar-endosomal route, but in contrast with other viruses are able to avoid lysosomal degradation, thereby evading exposure to the host immune system. SV40 has a 5.25 kb long circular double-stranded DNA possessing two genes. The early gene encodes two non-structural replication-associated proteins Small T antigen (STag) and Large T antigen (LTag). The late gene codes for the structural viral proteins VP1, VP2 and VP3.

The early and late genes are separated by the polyomaviral intergenic region that contains the early and late promoters needed for transcription of the early and late gene, the origin-of-replication needed for polyomaviral DNA replication and the packaging signal needed for the formation of polyomavirus particles.

Replication-defective SV40 vectors have been generated by deleting the coding region of the early gene leaving 2.7 kb of available space to clone exogenous DNA. In cells lacking the SV40 early gene transduced with the vector the absence of LTag prevents the production of all viral proteins. Because humans can be considered naïve to SV40, it is expected that replication-defective SV40 vectors are non-immunogenic when applied in humans. The non-immunogenicity in humans render SV40 vectors highly attractive for use in gene therapies to treat genetic disorders, cancer, autoimmune diseases and allergies, and to prevent transplant rejection.

Replication-defective SV40 vectors are produced in macaque packaging cell lines expressing the SV40 early gene such as COS-1, COT18 and CMT4, or in macaque cell lines expressing the SV40 early and late genes, such as COS-7. Packaging cell lines expressing the SV40 early and late genes can be used to produce replication defective SV40 vectors lacking the coding regions of the early and late genes. Such “gutless” vectors have a coding capacity of 4.8 kb of exogenous DNA (Mueller C et al., Gene Therapy 17: 227-237, 2010).

Passaging of SV40 vectors in said packaging cell lines however, results in the appearance of wild type SV40 particles. This most likely occurs by sequence homology-dependent recombination between the chromosomally inserted SV40-specific DNA sequences and episomally replicating SV40-specific DNA sequences.

To prevent the occurrence of replication-competent virus particles in the vector preparations, polyomavirus-based virus-like particle (VLP) vector systems have been developed. In vitro generated VLPs consisting of circular double-stranded polyomaviral vector DNA encapsidated with polyomaviral VP1. Such VLPs lack VP2 and VP3 in the capsids and histones covering the encapsidated DNA molecules. Although these particles display a higher packaging capacity, the absence of VP2/VP3 and histones in the VLPs has a negative impact on their transduction efficacy in vivo.

In order to overcome the generation of wild type virus contaminants during the production of replication-defective SV40 vector particles a safe and efficient Vero-based SV40 vector packaging cell line, named SuperVero, was generated. SuperVero cells solely express the viral LTag and accumulate fully replication-defective vector particles at high titers, comparable to those obtained in the conventional SV40 vector packaging cell lines (see for example: Toscano M G et al., Mol. Ther. Methods Clin. Dev. 6: 124-134, 2017; International patent application published under number WO 2010/122094 A1).

Currently, circular polyomaviral vector DNA required for starting the production of vector particles in packaging cell lines is generated by releasing the vector DNA from a plasmid backbone by restriction enzyme digestion followed by self-ligation of the resulting linear vector DNA using T4 DNA ligase to generate the circular polyomaviral vector genomes. A disadvantage of this method to generate circular vector DNA is that the circular DNA molecules have a relaxed conformity. Such relaxed circular vector DNA genomes are relatively poor substrates for the SV40 LTag to become replicated and packaged by SV40 capsid proteins to SV40 vector particles. To circumvent this problem circular polyomaviral vector DNA with a supercoiled conformity can be generated by introducing 2 recognition sequences for a tyrosine or serine DNA recombinase such as loxP sequences for Cre recombinase from bacteriophage T1, flanking the vector DNA in the polyomaviral vector plasmid. Addition of the tyrosine or serine DNA recombinase to the polyomaviral vector plasmid DNA in vitro results in the generation of circular supercoiled polyomaviral vector genomes. Alternatively, polyomaviral vector plasmid DNA containing the recognition sequences for a tyrosine or serine DNA recombinase is introduced in a polyomaviral vector packaging cells together with DNA encoding the cognate tyrosine or serine DNA recombinase resulting in the generation of circular supercoiled polyomaviral vector genomes in vivo (Shi X et al., Mol. Ther. Methods Clin. Dev. 9: 225-233, 2018).

A disadvantage of the replication-defective polyomaviral vectors with deleted coding regions of the early gene is the relatively limited space of 2.7 kb to clone exogenous DNA.

### DESCRIPTION OF THE INVENTION

In a first aspect, the present invention relates to a composition for use in the production of polyomaviral vector particles, the composition comprising:
a first DNA construct comprising recombinant DNA and a polyomaviral intergenic region, wherein said first DNA construct does not comprise polyomaviral functional coding sequences and bacterial plasmid sequences; and
a second DNA construct comprising polyomaviral functional coding sequences encoding functional polyomaviral capsid proteins, wherein said second DNA construct cannot be encapsidated into the polyomaviral vector particles.

As used herein, the term ‘polyomaviral intergenic region’ refers to the region of the polyomavirus vector DNA containing the early and late promoters needed for transcription of the early and late genes, the origin-of-replication needed for polyomaviral DNA replication and the packaging signal needed for the formation of polyomaviral vector particles.

It was found that by providing a first DNA construct not encoding polyomaviral functional proteins and bacterial plasmid sequences, and by providing a second DNA construct encoding functional polyomaviral capsid proteins, which second DNA construct cannot be encapsidated into the polyomaviral vector particles, not only a reliable and safe production of polyomaviral vector particles is provided. It was also found that the composition of the present invention provides in a high flexibility in designing the recombinant DNA to be used in the polyomaviral vector particles having an increased packaging capacity of the recombinant DNA compared to the currently used polyomaviral vector particles containing the polyomaviral late gene.

As stated above, the first DNA construct of the present invention does not comprise polyomaviral functional coding sequences and bacterial plasmid sequences. In other words, the first DNA construct of the present invention is substantially free of (e.g. does not comprise any) polyomaviral functional coding sequences, i.e. not encoding polyomaviral functional proteins, including functional polyomaviral capsid proteins (e.g. VP1, VP2 and VP3), replication-associated proteins Small T antigen (STag) and Large T antigen (LTag), agnoproteins and bacterial plasmid sequences.

It is noted that the term ‘first DNA construct’ as used herein may refer to similar terms used in the field including gutless vector, gutless vector plasmid or gutless vector DNA.

The actual size of the recombinant DNA comprised in the first DNA construct may vary, but is selected such that the recombinant DNA can be encapsidated into the polyomaviral vector particles. By providing a first DNA construct that does not comprise a sequence encoding a functional polyomaviral protein, the packaging capacity of the first DNA construct in relation to the recombinant DNA is increased. The present invention now provides for a first DNA construct, wherein the recombinant DNA may have a size of at least 3.0 kb. Typically, the recombinant DNA comprised in the first DNA construct may have a size between 4.0 kb and 6.0 kb.

It is noted that the term ‘recombinant DNA’ as used herein may refer to similar terms used in the field including DNA insert, transgene or transgene construct. The recombinant DNA may encode one or multiple therapeutic proteins or RNA molecules.

As stated above, the second DNA construct of the present invention encoding functional polyomaviral capsid proteins cannot be encapsidated into the polyomaviral vector particles. Preferably, the size of the second DNA construct is selected such that the second DNA construct cannot be encapsidated into the polyomaviral vector particles. Although the size of the second DNA construct may vary, preferably the second DNA construct may have a size of at least 10 kb, at least 11 kb, or more preferably a size of at least 12 kb.

As used herein, the term ‘second DNA construct’ may refer to a supporting vector genome or supporting DNA vector, i.e. a DNA construct or vector comprising DNA sequences, such as sequences encoding functional polyomaviral capsid proteins, facilitating the formation of polyomaviral vector particles.

Although the second DNA construct comprises polyomaviral functional coding sequences encoding functional polyomaviral capsid proteins (preferably selected from the group consisting of VP1, VP2 and VP3), in addition, the second DNA construct may also comprise polyomaviral functional coding sequences encoding a functional polyomaviral LTag. It was found that by providing a second DNA construct encoding functional polyomaviral capsid proteins and a functional polyomaviral LTag, the production of polyomaviral vector particles is no longer dependent on the additional functionality of the polyomaviral permissive cells in which the DNA constructs of the present invention are introduced.

The polyomaviral intergenic region and the polyomaviral functional coding sequences used in the constructs of the present invention are preferably derived from a primate polyomavirus, preferably a simian polyomavirus. Preferably the polyomaviral intergenic region and the polyomaviral functional coding sequences are derived from polyomaviruses selected from the group consisting of Simian virus 40, Macaca fascicularis polyomavirus 1, Pan troglodytes verus polyomavirus 1a, Pan troglodytes verus polyomavirus 2a, Pan troglodytes verus polyomavirus 3, Pan troglodytes verus polyomavirus 4, Pan troglodytes verus polyomavirus 8, Pan troglodytes schweinfurthii polyomavirus 2, Chimpanzee polyomavirus, Bornean orang utan polyomavirus, Sumatran orang utan polyomavirus or Gorilla gorilla gorilla polyomavirus 1, yellow baboon polyomavirus 1, yellow baboon polyomavirus 2, Vervet monkey polyomavirus 1, Vervet monkey polyomavirus 2, Vervet monkey polyomavirus 3 and Cercopithecus erythrotis polyomavirus 1. In a preferred embodiment, the polyomaviral intergenic region and the polyomaviral functional coding sequences are derived from macaque polyomavirus Simian Virus 40 (SV40).

The composition of the present invention may be a solution, e.g. an aqueous solution, preferably a physiological solution. The composition of the present invention may comprise the first DNA construct as a first vector and the second DNA construct as a different separate second vector. Preferably, the first DNA construct and second DNA constructed are comprised in the composition as a first plasmid and second plasmid, respectively. In addition, it is noted that in case both DNA constructs are provided as separate constructs, i.e. separate vectors or plasmids, it is preferred to provide a composition comprising an excess amount of first DNA construct and a limited amount of second DNA construct.

Alternatively, the first DNA construct and the second DNA construct of the composition of the present invention may be comprised in the same circular DNA. In other words, the composition comprises an amount, preferably an excess amount, of circular DNA, wherein each of the circular DNA is formed of a first DNA construct and a second DNA construct. In a preferred embodiment, the second DNA construct comprised on the circular DNA may further comprise a sequence encoding a recombinase enzyme, such as Cre recombinase.

The second DNA construct may be inserted in the chromosomal DNA of cells used for the production of polyomaviral vector particles of the invention.

In a second aspect, the present invention relates to a method for the production of polyomaviral vector particles, the method comprising the steps of:
a) providing a first DNA construct comprising recombinant DNA and a polyomaviral intergenic region, wherein said first DNA construct does not comprise polyomaviral functional coding sequences and bacterial plasmid sequences;
b) providing a cell line permissive for the wild type polyomavirus;
c) introducing the first DNA construct of step a) into the cell line of step b);
d) culturing the cell line obtained in step c) in a growth medium under conditions allowing the formation of polyomaviral vector particles; and
e) harvesting the polyomaviral vector particles from the cell culture obtained in step d),
wherein the method further comprises the step of introducing in step c) a second DNA construct capable of expressing functional polyomaviral capsid proteins, and wherein said second DNA construct cannot be encapsidated into the polyomaviral vector particles.

The second DNA construct comprises polyomaviral functional coding sequences encoding functional polyomaviral capsid proteins and, optionally, encoding a functional polyomaviral large T antigen.

The cell line permissive for the wild type polyomavirus as used in the method of the present invention and provided in step b) is preferably capable of expressing functional polyomaviral large T antigen. Preferably, the cell line permissive for the wild type polyomavirus is selected from the group consisting of Vero, CV1 or BSC-1 cells or derivatives thereof.

Further, it is noted that the first DNA construct comprising recombinant DNA and the polyomaviral intergenic region is capable of replication in said cell line permissive for the wild type polyomavirus.

Both DNA constructs may be introduced into the cell line in step c) as separate DNA constructs, wherein the first DNA construct provided in step a) is comprised in a circular DNA and/or wherein the second DNA construct is comprised in a circular DNA.

Alternatively, both DNA constructs may be provided as a single circular DNA comprising the first DNA construct and the second DNA construct. In such embodiment, the single circular DNA is introduced into the cell line in step c).

In a third aspect, the present invention relates to a composition comprising polyomaviral vector particles obtainable by the method of the present invention. It is noted that the polyomaviral vector particles obtainable by the method of the present invention are polyomaviral vector particles not encoding functional polyomaviral proteins, such as functional polyomaviral capsid proteins and functional polyomaviral replication-associated proteins, and bacterial plasmid sequences. It is further noted that the polyomaviral vector particles obtainable by the method of the present invention being incapable of replication in cells permissive for the wildtype polyomavirus. In particular, the present invention relates to a composition that does not contain a single polyomavirus particle being capable of replicating in cells permissive for the wildtype polyomavirus wherein said cells do not express functional polyomaviral large T antigen and functional polyomaviral capsid proteins.

In a preferred embodiment, the present invention relates to a composition comprising more than one million polyomaviral vector particles obtainable by the method of the present invention.

In a fourth aspect, the present invention relates to polyomaviral vector particles obtainable by the method of the present invention for use as a medicament. Preferably, the present invention relates to polyomaviral vector particles obtainable by the method of the present invention for use in the treatment of genetic disorders and immunity-associated diseases including degenerative diseases, inflammatory diseases, autoimmune diseases, allergies, cancer and in the treatment of transplant rejection.

### EXAMPLES

### Construction of the Dual Gutless MaxVec Vector System

The MaxVec dual replicon gutless vector system was constructed using two previously described plasmids: pSVac (Toscano M et al., 2017), which is a plasmid that encodes the SV40 intergenic region and the late region; and pHY359, which is a pBluescript-based plasmid that encodes the SV40 LTag under transcriptional control of the EIF1a promoter.

The MaxVec vector plasmid pMaxVec (pAM467; SEQ ID NO: 1; see also: FIG. 1A) was constructed by removing the late region from pSVac. Subsequently, a gateway recombination DNA cassette was inserted to facilitate cloning of transgenes by Gateway recombination (ThermoFisher Scientific). A multiple cloning site was added downstream of the SV40 late promoter to allow the addition of transgene DNA in this case “filler” DNA (pAM615; SEQ ID NO: 2; see also: FIG. 1B).

Here it was noted that the addition of filler DNA can be important if a transgene is used with a size that does not result in the formation of vector particles.

Furthermore, two LoxP recombination sites were added that flank the viral genes within the plasmid. This enables the exclusion of the bacterial backbone that contains the ampicillin resistance gene and the bacterial origin of replication, from the viral vector using the enzyme Cre-recombinase.

An SV40 MaxVec helper plasmid (pAM560; SEQ ID NO: 3; see also: FIG. 1C) was constructed, as MaxVec vector particles cannot be produced in cells lacking SV40 LTag and capsid proteins. This helper plasmid is built by adding a blasticidin resistance gene behind the SV40 early promoter and the SV40 LTag gene under transcriptional control of the EF1-alpha promoter from pHY359. The EF1-alpha-Ltag sequence was inserted downstream of the blasticidin resistance gene. The SV40 late region was kept under transcriptional control of the SV40 late promoter.

### Testing of the Dual Gutless MaxVec Vector System

To test the newly constructed MaxVec dual vector system. SuperVero cells were co-transfected in a 1:1 ratio with helper plasmid pAM560 and a Cre-recombined (New England Biolabs) MaxVec plasmid that encodes the firefly luciferase (MaxVLuc) or hrGFPII reporter gene (MaxVGFP) as the transgene. Here cells were seeded at a density of 10,000 cells/cm² the day before transfection. After transfection using polyethyleneimine (PEI) with a 4:1 PEI:DNA weight to weight ratio the cells were incubated at 37° C. overnight and were washed using optipro medium the next day. Three days post transfection, the supernatant, and cells were collected separately. The MaxVLuc-containing cells were lysed in accordance with manufacturer's instructions (Promega) and the firefly luciferase luminescence was determined (Glomax, Promega). The MaxVLuc-containing supernatant was incubated with fresh SuperVero cells that were seeded at a density of 10,000 cells/cm². Three days later the transduced cells were inspected for the presence of GFP by fluorescence microscopy.

Results of the transfection experiments demonstrated that the SV40 early promoter remains active and drives transgene expression (see: FIG. 2 and FIG. 3). Furthermore, luminescence could be detected in the cells that were transduced using the supernatant of the transfected cells, indicating that MaxVec vector were produced in the transfected SuperVero cells. These results demonstrate that the MaxVec dual replicon vector system provides potent MaxVec vector particles that are capable of transducing cells and expressing the transgene in transduced cells.

### Optimalisation of MaxVec Particle Production

Different transgene sequence lengths were tested to identify the optimal genome size for producing MaxVec particles in SuperVero cells. Multiple MaxVec constructs were made, each encoding the firefly luciferase together with different lengths of non-coding “filler” DNA. The resulting MaxVec plasmids were cre-recombined to generate circular vector DNA molecules. SuperVero cells were co-transfected with the MaxVec filler DNA variants and the helper plasmid. Supernatant was harvested 3 days post-transfection. To ascertain the transduction efficiency/capacity of the various MaxVec particles, the supernatant was subsequently added to fresh Vero and SuperVero cells seeded at 5×10³ cells/cm². The transfected cells were incubated for 3 days and the number of vector particles that were produced in the transfected cells was measured by luminescence using the dual-luciferase assay.

Results demonstrated (see: FIG. 4) that a vector size of up to 6.0 kb can be packaged. A genome length between 4.0 kb and 6 kb proved to result in generating particles that were capable of transducing SuperVero cells most efficiently.

### MaxVec Production Generates Replication Deficient Particles

MaxVec particles, encoding firefly luciferase (MaxVLuc), were produced in SuperVero cells using the pAM486 helper plasmid (SEQ ID NO: 4; see also: FIG. 1D). The particles were collected at day 3 and day 6 post transfection from the supernatant, measured and pooled. SuperVero cells were seeded and the MaxVLuc particles were added to the cells. Subsequently, supernatant from the transduced SuperVero cells was collected, and luminescence was measured by isolating the cells at 7 days post transduction. Using the dual-luciferase assay (Promega), luminescence was measured. Hereafter the supernatant was again added to fresh SuperVero cells and the procedure was repeated for another 2 rounds. The cells that were either initially transfected or transduced demonstrated luminescence. SuperVero cells that were transduced with supernatant that was collected from the first or later transduction attempts did not show luminescence (see: FIG. 5). These data indicate that MaxVec particles cannot replicate in cells that do not contain the SV40 LTag and capsid proteins. Therefore, MaxVec particles cannot be produced in SuperVero cells lacking the SV40 capsid proteins.

### BRIEF DESCRIPTION OF THE DRAWINGS

FIG. 1A-D depicts the vector map of the plasmids pAM467, pAM615, pAM560 and pAM486, respectively.

FIG. 2 shows the results of SuperVero cells transduced with MaxVec particles encoding GFP (MaxVGFP). MaxVGFP particles were produced in SuperVero cells using pAM486 as a helper plasmid. Three days post transfection, supernatant was harvested and added to fresh SuperVero cells. GFP+ signal indicates transduced cells. A: SVGFP (control), B: MaxVGFP (4.7 kB) and C: negative cell control. Images 1 show the cells while images 2 show the fluorescence signals. Observed under a fluorescence microscope (509 nm) 3 days post-transduction at 100× magnification.

FIG. 3 shows the results of MaxVLuc transduced SuperVero cells. MaxVLuc particles were produced in SuperVero cells using pAM486 as a helper plasmid. Luminescence of firefly luciferase was measured 3 days post transduction in triplicate. The bars represent a single transduction. The SVLuc control is based on the SVec vector that has the SV40 late gene.

FIG. 4 shows firefly luciferase expression of pSVLuc as a control and MaxVLuc vectors having different genome lengths. SuperVero cells were transfected with the different vector DNAs with genome lengths varying from 2.5 to 6.0 kb, with or without BGH poly-adenylation sequence downstream of the SV40 late promoter. Supernatants of the transfected cells were collected and subsequently added to fresh SuperVero cells in triplicate (n=3). Three days post transduction, firefly luciferase was measured. TRF=firefly luciferase signal after transfection of SuperVero cells as a measure of the transfection efficiency, TRD=firefly luciferase signal after transduction of SuperVero cells as a measure of MaxVec potency. MaxVec vectors with a genome length of 4.0-6.0 kb are produced at the highest titers.

FIG. 5 shows serial passaging of SVLuc versus MaxVLuc in SuperVero cells. SuperVero cells were used to produce MaxVLuc and SVLuc particles after transfection (TRF) of cre-recombined plasmid DNAs. Supernatant from the cells was collected and added to fresh SuperVero cells and serially passaged to fresh SuperVero cells for 3 passages. Luminescence was measured after the 4^{th} transduction (TRD) cycle. The measurements indicated that the SuperVero cells transduced with SVLuc still produce vector particles. However, the luminescence dropped after the first passage for MaxVLuc, indicating that MaxVec particles were generated after transfection of SuperVero with cre-recombined plasmid DNA but not after transduction of SuperVero cells.

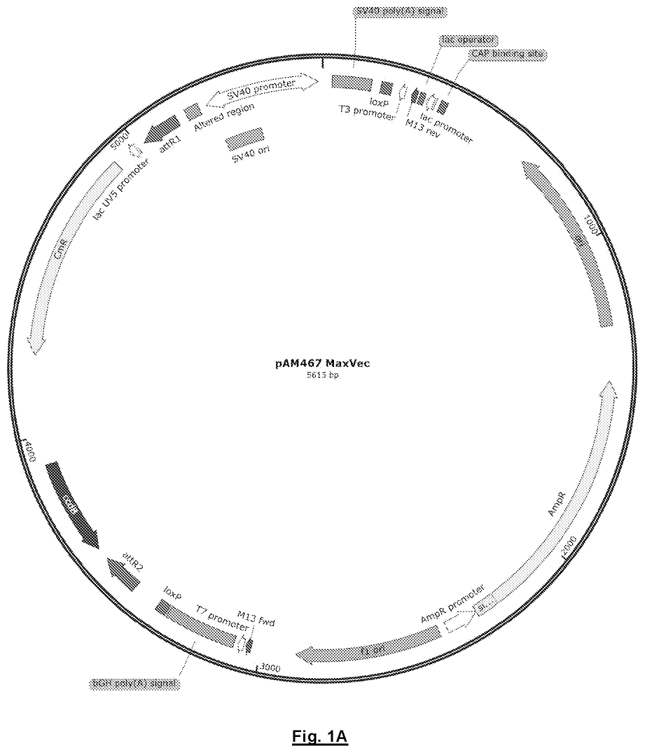

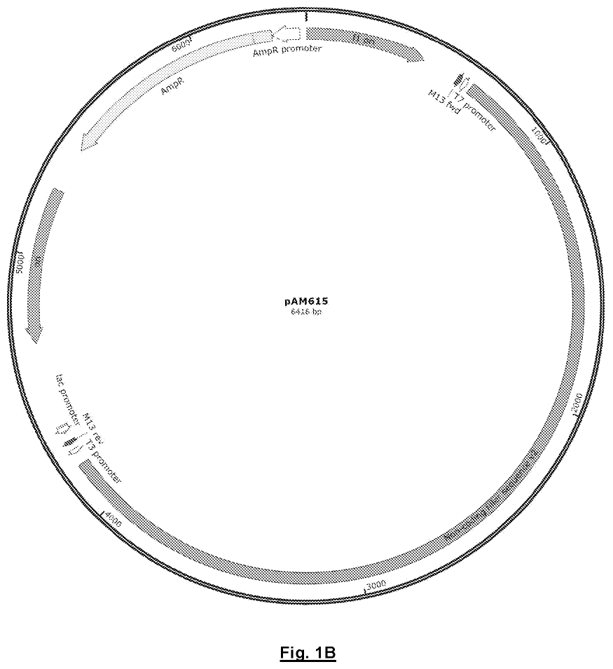

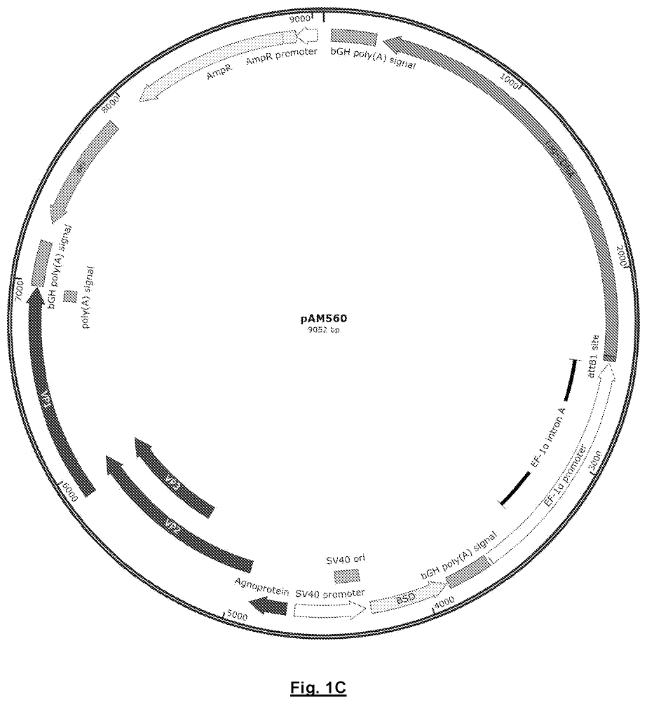

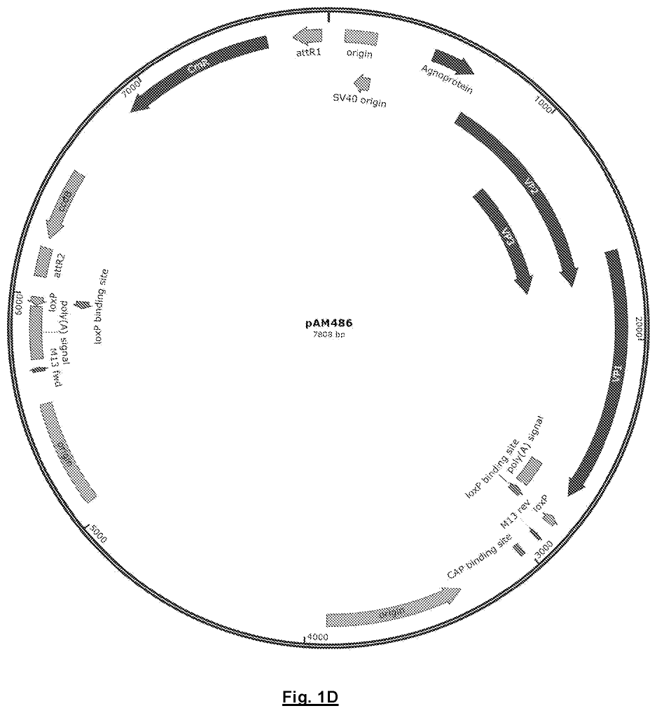

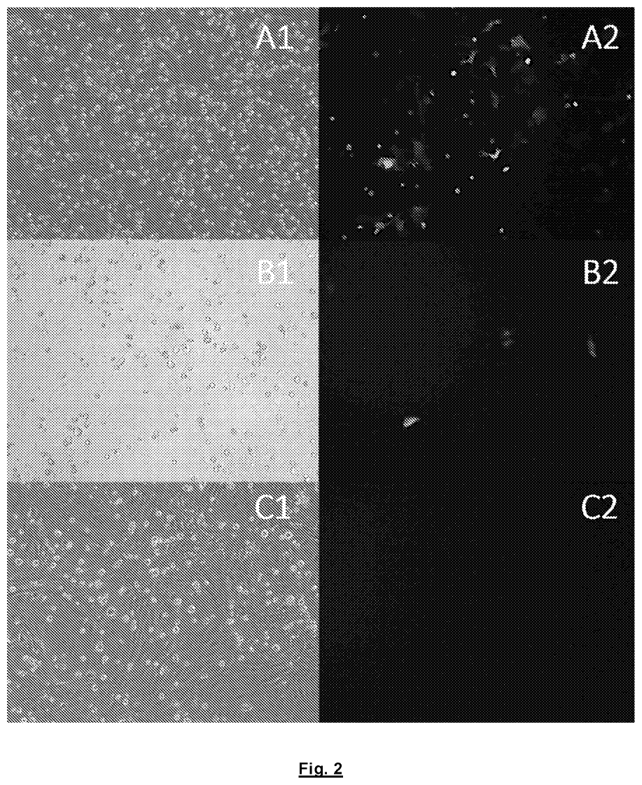

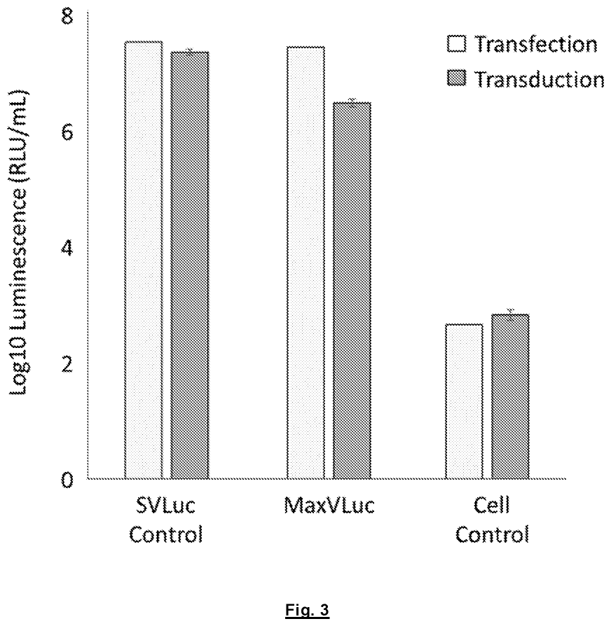

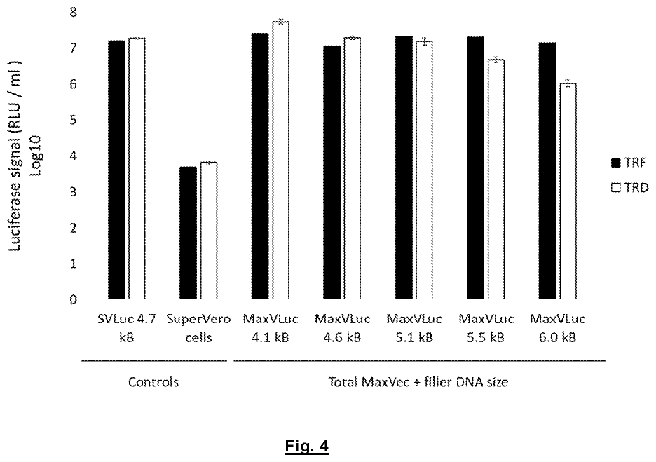

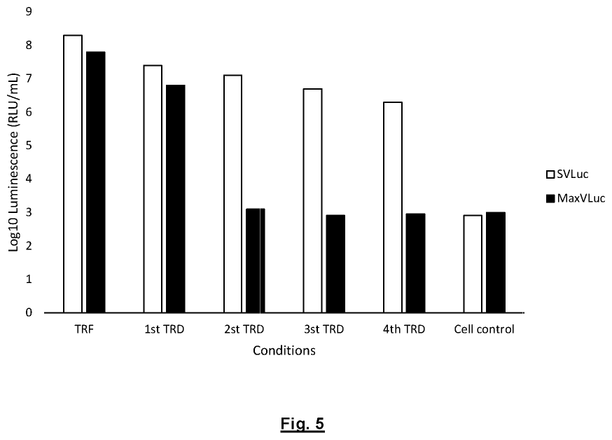

1. A composition comprising:
a first DNA construct comprising recombinant DNA and a polyomaviral intergenic region, wherein said first DNA construct does not comprise polyomaviral functional coding sequences and bacterial plasmid sequences; and
a second DNA construct comprising polyomaviral functional coding sequences encoding functional polyomaviral capsid proteins, wherein said second DNA construct cannot be encapsidated into the polyomaviral vector particles to be produced.

2. The composition according to claim 1, wherein the size of the recombinant DNA is selected such that the recombinant DNA can be encapsidated into the polyomaviral vector particles.

3. The composition according to claim 1, wherein the size of the second DNA construct is selected such that the second DNA construct cannot be encapsidated into the polyomaviral vector particles.

4. The composition according to claim 1, wherein the second DNA construct encodes a functional polyomaviral large T antigen.

5. The composition according to claim 1, wherein the polyomaviral intergenic region and the polyomaviral functional coding sequences are derived from a primate polyomavirus.

6. The composition according to claim 1, wherein the first DNA construct is a first vector, wherein the second DNA construct is a second vector.

7. The composition according to claim 1, wherein the first DNA construct and the second DNA construct are comprised in the same circular DNA.

8. The composition according to claim 7, wherein the second DNA construct comprised on the circular DNA further comprises a sequence encoding a recombinase enzyme.

9. A method for the production of polyomaviral vector particles, the method comprising the steps of:
a) providing a first DNA construct comprising recombinant DNA and a polyomaviral intergenic region, wherein said first DNA construct does not comprise polyomaviral functional coding sequences and bacterial plasmid sequences;
b) providing a cell line permissive for the wild type polyomavirus;
c) introducing the first DNA construct of step a) into the cell line of step b);
d) culturing the cell line obtained in step c) in a growth medium under conditions allowing the formation of polyomaviral vector particles; and
e) harvesting the polyomaviral vector particles from the cell culture obtained in step d),
wherein the method further comprises the step of introducing in step c) a second DNA construct capable of expressing functional polyomaviral capsid proteins, and wherein said second DNA construct cannot be encapsidated into the polyomaviral vector particles.

10. The method according to claim 9, wherein the cell line permissive for the wild type polyomavirus provided in step b) is capable of expressing functional polyomaviral large T antigen.

11. The method according to claim 9, wherein the first DNA construct provided in step a) is comprised in a circular DNA, wherein the circular DNA further comprises the second DNA construct comprising polyomaviral functional coding sequences encoding functional polyomaviral capsid proteins and, optionally, encoding a functional polyomaviral large T antigen.

12. A composition comprising water and polyomaviral vector particles obtainable by the method of claim 9.

13. Polyomaviral vector particles prepared according to the method of claim 9.

14. A method of treating a genetic disorder or immunity-associated disease in a subject, the method comprising: administering polyomaviral vector particles prepared according to the method of claim 9 to a subject in need thereof, thereby treating the genetic disorder or immunity-associated disease in the subject.

15. The method of claim 14, wherein the genetic disorder or immunity-associated disease is a degenerative disease, inflammatory disease, autoimmune disease, allergy, cancer, or transplant rejection.

16. The composition according to claim 2, wherein the recombinant DNA has a size of at least 3.0 kb.

17. The composition according to claim 2, wherein the recombinant DNA has a size between 4.0 kb and 6.0 kb.

18. The composition according to claim 5, wherein the primate polyomavirus is a simian polyomavirus.

19. The composition according to claim 18, wherein the simian polyomavirus is macaque polyomavirus Simian Virus 40 (SV40).

20. The composition according to claim 8, wherein the recombinase enzyme is a Cre recombinase.
